(19) United States
(12) Patent Application Publication
(10) Pub. No.: US 2004/0115153 A1
(43) Pub. Date: Jun. 17, 2004

(54) Compositions containing at least one oil structured with at least one silicone-polyamide polymer, and at least one silicone gum and methods of using the same

(75) Inventor: Wei Yu, Edison, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/320,599

(22) Filed: Dec. 17, 2002

(51) Int. Cl. A61K 7/025; A61K 7/06; A61K 7/11

(52) U.S. Cl. 424/70.12

(57) ABSTRACT

The invention relates to a physiologically acceptable composition, in particular a cosmetic composition, containing at least one liquid fatty phase structured with at least one structuring polymer of the silicone-polyamide type, the polymer being solid at room temperature and soluble in the liquid fatty phase at a temperature of from 25 to 250° C., and at least one silicone gum, said oil having an affinity with said structuring polymer and/or with said silicone gum, and the liquid fatty phase, the polymer and the silicone gum forming a physiologically acceptable medium. This composition may be in the form of a stick of lipstick which is stable, which does not exude and whose application produces a glossy deposit with good staying power over time.

The present invention relates to a care and/or treatment and/or make-up composition for the skin, including the scalp, and/or for the lips of human beings, and/or for keratinous materials, such as keratinous fibers, containing a liquid fatty phase, structured with a specific polymer and comprising silicone gums.

This composition can be stable over time and may be in the form of a tube or stick of make-up such as lipstick, or in the form of a paste such a lip balm, the application of which can produce a non tacky, light feel and long-wearing properties.

It is common to find a structured, i.e., gelled and/or rigidified, liquid fatty phase in cosmetic or dermatological products; this is especially the case in solid compositions such as deodorants, lip balms, lipsticks, concealer products, eye shadows and cast foundations. This structuring may be obtained with the aid of waxes and/or fillers. Unfortunately, these waxes and fillers may have a tendency to make the composition matte, which may not always be desirable, in particular for a lipstick or an eye shadow. Consumers are always on the lookout for a lipstick in stick form which can deposit a film with good staying power or long wearing properties but which is also increasingly glossy.

The structuring of the liquid fatty phase may make it possible in particular to limit its exudation (or syneresis) from solid compositions, particularly in hot and humid areas and, furthermore, after deposition on the skin or the lips, to limit the migration of this phase into wrinkles and fine lines, a characteristic particularly desirable in a lipstick or eye shadow. The reason for this is that considerable migration of the liquid fatty phase, particularly when it is charged with coloring agents, may lead to an unpleasant appearance around the lips and the eyes, making wrinkles and fine lines particularly prominent. Consumers often state this migration as being a major drawback of conventional lipsticks and eye shadows. The term “migration” means movement of the composition beyond its initial site of application.

Gloss of a lipstick or other cosmetic is generally associated with the nature of the liquid fatty phase. Thus, it may be possible to reduce the amount of waxes and/or fillers in the composition in order to increase the gloss of a lipstick, but in that case the migration of the liquid fatty phase may increase. In other words, the amounts of waxes and of fillers required to prepare a stick of suitable hardness that does not exude at room temperature are a restricting factor on the gloss of the deposit.

To overcome at least one of these drawbacks, it has been envisaged replacing all or some of the waxes and/or fillers with polymers for structuring the liquid fatty phase, of the silicone-polyamide type. Unfortunately, the sticks obtained are not mechanically or thermally stable.

Furthermore, make-up compositions should have good staying power or long-wearing properties over time, i.e., little turning of or change in color over time or a gradual or homogeneous change of the deposit over time. The turning of or change in color of the deposit may be due, for lipsticks, to an interaction with saliva and, for foundations and eye shadows, to an interaction with the sweat and sebum secreted by the skin. Make-up compositions should not transfer, that means the compositions should not transfer at least partially, while leaving a stain, on some supports that they come in contact with, such as a glass, a cup, an article of clothing or even skin. This results in a mediocre wear of the film on the skin or the lips, thus requiring the user to frequently reapply the composition on the skin or the lips.

The need thus remains for a composition which does not have at least one of the above drawbacks, which has good stability over time, even in hot atmosphere, and which produces a deposit on the skin or the lips that shows good staying power or long-wearing over time and has a glossy appearance. Furthermore, this composition can be easy to manufacture and can give the deposit a sensation of not drying out, both during application and over time.

One subject of the invention is a care and/or make-up and/or treatment composition for the skin and/or the lips of the face and/or for superficial body growths, i.e., keratinous materials, such as nails or keratinous fibers, which makes it possible to overcome at least one of the drawbacks mentioned above.

The inventors have found, surprisingly, that the use of at least one specific structuring polymer combined with a compatible fatty phase and at least one silicone gum makes it possible to obtain a stick whose application to the lips produces a deposit which can have noteworthy cosmetic properties.

In particular, the deposit can be at least one of glossy, supple, comfortable and “migration-resistant”. Furthermore, the composition can be stable over time at room temperature (25° C.) as well as high temperature (typically 47° C.).

The term “stable” refers to a composition, in particular a stick, that is hard, and does not collapse over time at room temperature (25° C.) and at 47° C. for at least 1 month

The invention applies not only to make-up products for the lips, such as lipsticks, lip glosses and lip pencils, but also to care and/or treatment products for the skin, including the scalp, and for the lips, such as antisun care products for the human face, the body or the lips, such as in stick form, make-up removing products for the skin of the face and body, make-up products for the skin, both of the human face and body, such as foundations optionally cast in stick or dish form, concealer products, blushers, eyeshadows, face powders, transfer tattoos, body hygiene products (i.e., products which do not relate to the care, make-up, or treatment of keratin materials) such as deodorant, e.g., in stick form, shampoos, conditioners and make-up products for the eyes such as eyeliners, eye pencils and mascaras, e.g., in cake form, as well as make-up and care products for superficial body growths, for instance keratinous fibers such as the hair, the eyelashes, and the eyebrows or nails.

Another aspect of the invention is a composition comprising at least one liquid fatty phase comprising (i) at least one oil structured with at least one structuring polymer consisting of a polymer (homopolymer or copolymer) with a weight-average molecular mass ranging from 500 to 500 000, containing at least one moiety comprising:

at least one polyorganosiloxane group, consisting of from 1 to 1 000 organosiloxane units in the chain of the moiety or in the form of a graft, and

at least two groups capable of establishing hydrogen interactions, chosen from ester, amide, sulphonamide, carbamate, thiocarbamate, urea, urethane, thiourea, oxamido, guanamido and biguanidino groups, and combinations thereof,

the polymer being solid at 25° C. and soluble in the liquid fatty phase at a temperature of from 25 to 250° C., and

(ii) at least one silicone gum,

said oil having an affinity with said structuring polymer and/or with the silicone gum, and the liquid fatty phase, the polymer and the silicone gum forming a physiologically acceptable medium.

As used herein, the expression “at least one” means one or more and thus includes individual components as well as mixtures/combinations.

The polymer structuring the liquid fatty phase is solid at room temperature (25° C.) and atmospheric pressure (760 mm Hg) and soluble in the liquid fatty phase at a temperature of from 25 to 250° C.

As used herein, the expression “polymer” means a compound having at least two repeating units, preferably at least three repeating unit, more preferably at least ten repeating unit. In the composition according to the present invention, the structuring polymer represents 0.5 to 80% by weight, preferably 2 to 60% by weight, more preferably 5 to 40% by weight, of the total weight of the composition.

Moreover, the structuring polymer preferably represents 2 to 40% by weight of the weight of the silicone gum together with the oil included in the liquid fatty phase.

The liquid fatty phase preferably contains at least 40%, and better still at least 50% by weight of silicone oil.

The composition of the invention can be in the form of a paste, a solid or a more or less viscous cream. It can be a single or multiple emulsion, such as an oil-in-water or water-in-oil emulsion or an oil-in-water-in-oil emulsion, or a water-in-oil-in-water emulsion, or a rigid or soft gel containing an oily continuous phase. For example, the liquid fatty phase can be the continuous phase of the composition. In one embodiment, the composition is anhydrous. In one embodiment, the composition is in a form cast as a stick or in a dish, for example solid, and further example, in the form of an oily rigid gel, such as an anhydrous gel, e.g., an anhydrous stick. In a further embodiment, the composition is in the form of an opaque or translucent rigid gel (depending on the presence or absence of pigments), and in a specific example, the liquid fatty phase forms the continuous phase. In one embodiment, the composition is chosen from molded and poured sticks.

The structuring of the liquid fatty phase can be modified depending on the nature of the structuring polymer and the silicone gum that are used, and may be such that a rigid structure in the form of a stick is obtained. When these sticks are colored, they make it possible, after application, to obtain a uniformly colored and glossy deposit which does not migrate and/or which has good staying power, in particular of the color over time.

The composition of the invention can be a composition for the lips, such as a lipstick composition in stick form.

The polymers used as structuring agents in the composition of the invention are polymers of the polyorganosiloxane type such as those described in documents U.S. Pat. No. 5,874,069, U.S. Pat. No. 5,919,441, U.S. Pat. No. 6,051,216, and U.S. Pat. No. 5,981,680.

The structuring polymers to which the invention applies are solids that may be dissolved beforehand in a solvent with hydrogen interaction capable of breaking the hydrogen interactions of the polymers, for instance C₂ to C₈ lower alcohols and especially ethanol, n-propanol or isopropanol, before being placed in the presence of the silicone oils according to the invention. It is also possible to use these hydrogen interaction “breaking” solvents as co-solvents. These solvents may then be stored in the composition or may be removed by selective evaporation, which is well known to those skilled in the art.

The polymers comprising two groups capable of establishing hydrogen interactions in the polymer chain may be polymers comprising at least one moiety corresponding to the formula:

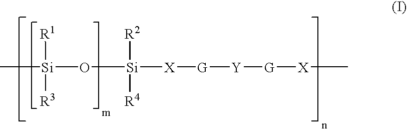

in which:

1) R¹, R², R³ and R⁴, which may be identical or different, represent a group chosen from:

linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,

C₆ to C₁₀ aryl groups, optionally substituted with one or more C₁ to C₄ alkyl groups,

polyorganosiloxane chains possibly containing one or more oxygen, sulphur and/or nitrogen atoms;

2) the groups X, which may be identical or different, represent a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:

fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or

4) Y represents a group corresponding to the formula:

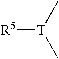

in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁵ represents a linear or branched C₁ to C₅₀ alkyl group or a polyorganosiloxane chain, possibly comprising one or more ester, amide, urethane, thiocarbamate, urea, urethane, thiourea and/or sulphonamide groups, which may be linked to another chain of the polymer;

5) the groups G, which may be identical or different, represent divalent groups chosen from:

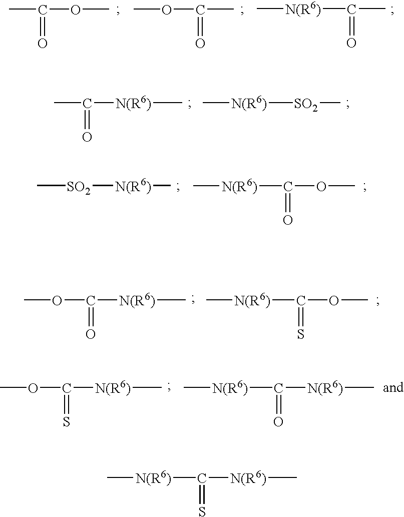

in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than:

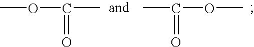

6) n is an integer ranging from 2 to 500 and preferably from 2 to 200, and m is an integer ranging from 1 to 1 000, preferably from 1 to 700 and better still from 6 to 200.

According to the invention, 80% of the groups R¹, R², R³ and R⁴ of the polymer are preferably chosen from methyl, ethyl, phenyl and 3,3,3-trifluoropropyl groups.

According to the invention, Y can represent various divalent groups, furthermore optionally comprising one or two free valencies to establish bonds with other moieties of the polymer or copolymer. Preferably, Y represents a group chosen from:

a) linear C₁ to C₂₀ and preferably C, to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C, to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C, to C₆ alkylamine groups,

g) polyorganosiloxane chains of formula:

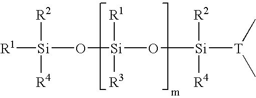

in which R¹, R², R³, R⁴, T and m are as defined above, and

h) polyorganosiloxane chains of formula:

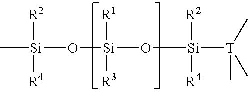

in which R¹, R², R³, R⁴, T and m are as defined above.

The polyorganosiloxanes of the second family may be polymers comprising at least one moiety corresponding to formula (II):

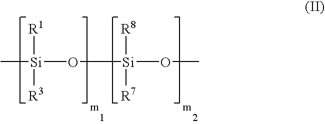

in which

R¹ and R³, which may be identical or different, are as defined above for formula (I),

R⁷ represents a group as defined above for R¹ and R³, or represents a group of formula —X-G-R⁹ in which X and G are as defined above for formula (I) and R⁹ represents a hydrogen atom or a linear, branched or cyclic, saturated or unsaturated, C, to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R⁸ represents a group of formula —X-G-R⁹ in which X, G and R⁹ are as defined above,

m₁ is an integer ranging from 1 to 998, and

m₂ is an integer ranging from 2 to 500.

According to the invention, the polymer used as structuring agent may be a homopolymer, that is to say a polymer comprising several identical moieties, in particular moieties of formula (I) or of formula (II).

According to the invention, it is also possible to use a polymer consisting of a copolymer comprising several different moieties of formula (I), that is to say a polymer in which at least one of the groups R¹, R², R³, R⁴, X, G, Y, m and n is different in one of the moieties. The copolymer may also be formed from several moieties of formula (II), in which at least one of the groups R¹, R³, R⁷, R⁸, m₁ and m₂ is different in at least one of the moieties.

It is also possible to use a copolymer comprising at least one moiety of formula (I) and at least one moiety of formula (II), the moieties of formula (I) and the moieties of formula (II) possibly being identical to or different from each other.

According to one variant of the invention, it is also possible to use a copolymer furthermore comprising at least one hydrocarbon-based moiety comprising two groups capable of establishing hydrogen interactions, chosen from ester, amide, sulphonamide, carbamate, thiocarbamate, urea, urethane, thiourea oxamido, guanamido and biguanidino groups, and combinations thereof.

These copolymers may be block copolymers or grafted copolymers.

According to a first embodiment of the invention, the groups capable of establishing hydrogen interactions are amide groups of formulae —C(O)NH— and —HN—C(O)—.

In this case, the structuring agent may be a polymer comprising at least one moiety of formula (III) or (IV):

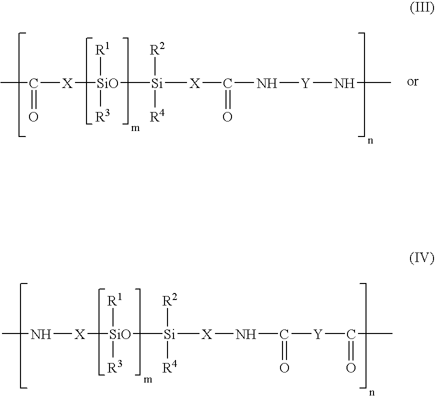

in which R¹, R², R³, R⁴, X, Y, m and n are as defined above.

Such a moiety may be obtained:

either by a condensation reaction between a silicone containing α,ω-carboxylic acid ends and one more diamines, according to the following reaction scheme:

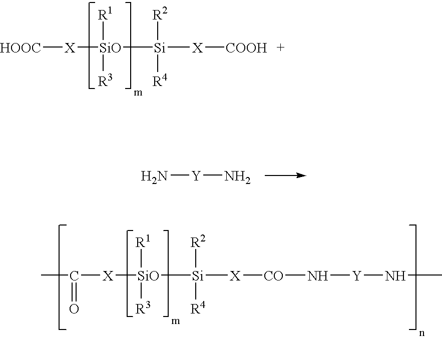

or by reaction of two molecules of α-unsaturated carboxylic acid with a diamine according to the following reaction scheme:

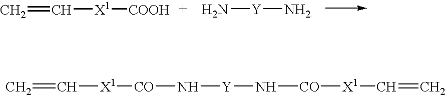

followed by the addition of a siloxane to the ethylenic unsaturations, according to the following scheme:

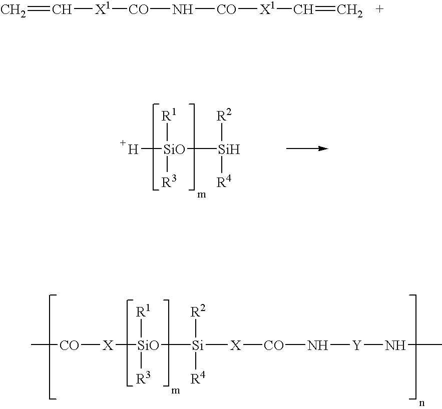

in which X¹—(CH₂)₂— corresponds to X defined above and Y, R¹, R², R³, R⁴ and m are as defined above;

or by reaction of a silicone containing α,ω—NH₂ ends and a diacid of formula HOOC—Y—COOH according to the following reaction scheme:

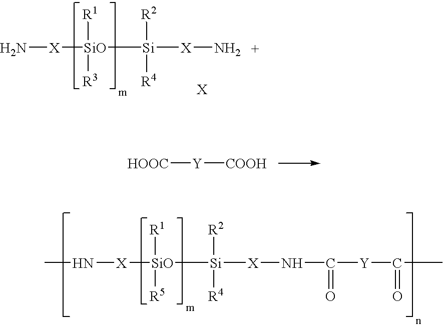

In these polyamides of formula (III) or (IV), m is preferably in the range from 1 to 700, more preferably from 15 to 500 and better still from 15 to 45, and n is in particular in the range from 1 to 500, preferably from 1 to 100 and better still from 4 to 25,

X is preferably a linear or branched alkylene chain containing from 1 to 30 carbon atoms and in particular 3 to 10 carbon atoms, and

Y is preferably an alkylene chain that is linear or branched or that possibly comprises rings and/or unsaturations, containing from 1 to 40 carbon atoms, in particular from 1 to 20 carbon atoms and better still from 2 to 6 carbon atoms, in particular 6 carbon atoms.

In formulae (III) and (IV), the alkylene group representing X or Y can optionally contain in its alkylene portion at least one of the following elements:

1°) 1 to 5 amide, urea or carbamate groups,

2°) a C₅ or C₆ cycloalkyl group, and

3°) a phenylene group optionally substituted with 1 to 3 identical or different C₁ to C₃ alkyl groups.

In formulae (III) and (IV), the alkylene groups may also be substituted with at least one element chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group.

In these formulae (III) and (IV), Y may also represent:

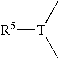

in which R⁵ represents a polyorganosiloxane chain and T represents a group of formula:

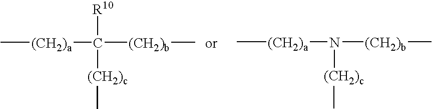

in which a, b and c are, independently, integers ranging from 1 to 10, and R¹⁰ is a hydrogen atom or a group such as those defined for R¹, R², R³ and R⁴.

In formulae (III) and (IV), R¹, R², R³ and R⁴ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different moieties of formula (III) or (IV).

Thus, the polymer may be a polyamide containing several moieties of formula (III) or (IV) of different lengths, i.e. a polyamide corresponding to the formula:

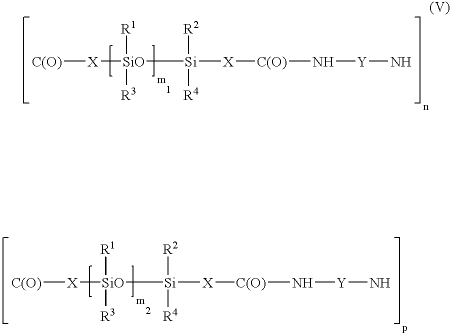

in which X, Y, n and R¹ to R⁴ have the meanings given above, m₁ and m₂, which are different, are chosen in the range from 1 to 1 000, and p is an integer ranging from 2 to 300. In this formula, the moieties may be structured to form either a block copolymer, or a random copolymer or an alternating copolymer. In this copolymer, the moieties may be not only of different lengths, but also of different chemical structures, for example containing different groups Y. In this case, the copolymer may correspond to the formula:

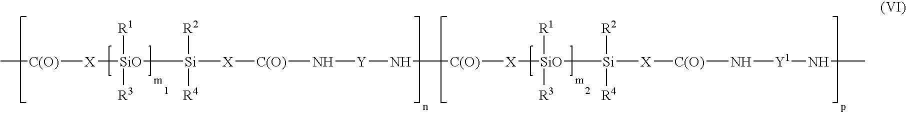

in which R¹ to R⁴, X, Y, m₁, m₂, n and p have the meanings given above and Y¹ is different from Y but chosen from the groups defined for Y. As previously, the various moieties may be structured to form either a block copolymer, or a random copolymer or an alternating copolymer.

In this first embodiment of the invention, the structuring polymer may also consist of a grafted copolymer. Thus, the polyamide containing silicone units may be grafted and optionally crosslinked with silicone chains containing amide groups. Such polymers may be synthesized with trifunctional amines.

In this case, the copolymer may comprise at least one moiety of formula:

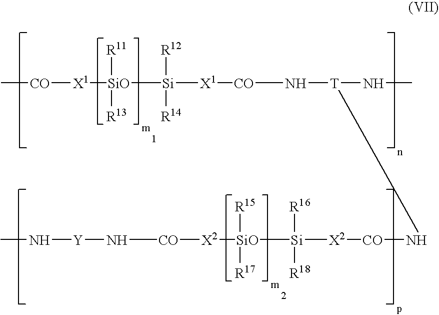

in which X¹ and X², which may be identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

In formula (VII), it is preferred that:

p is in the range from 1 to 25 and better still from 1 to 7,

R¹¹ to R¹⁸ are methyl groups,

T corresponds to one of the following formulae:

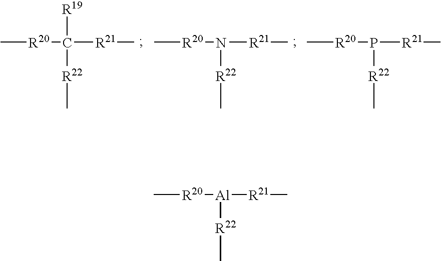

in which R¹⁹ is a hydrogen atom or a group chosen from the groups defined for R¹ to R⁴, and R²⁰, R²¹ and R²² are, independently, linear or branched alkylene groups, and more preferably corresponds to the formula:

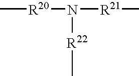

in particular with R²⁰, R²′ and R²² representing —CH₂—CH₂—,

m₁ and m₂ are in the range from 15 to 500 and better still from 15 to 45,

X¹ and X² represent —(CH₂)₁₀—, and

Y represents —CH₂—.

These polyamides containing a grafted silicone moiety of formula (VII) may be copolymerized with polyamide-silicones of formula (II) to form block copolymers, alternating copolymers or random copolymers. The weight percentage of grafted silicone moieties (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to the invention, the preferred siloxane-based polyamides are:

polyamides of formula (III) in which m is from 15 to 50;

mixtures of two or more polyamides in which at least one polyamide has a value of m in the range from 15 to 50 and at least one polyamide has a value of m in the range from 30 to 50;

polymers of formula (V) with m₁ chosen in the range from 15 to 50 and m₂ chosen in the range from 30 to 500 with the portion corresponding to m₁ representing 1% to 99% by weight of the total weight of the polyamide and the corresponding portion m₂ representing 1% to 99% by weight of the total weight of the polyamide;

mixtures of polyamide of formula (III) combining

1) 80% to 99% by weight of a polyamide in which n is equal to 2 to 10 and in particular 3 to 6, and

2) 1% to 20% of a polyamide in which n is in the range from 5 to 500 and in particular from 6 to 100;

polyamides corresponding to formula (VI) in which at least one of the groups Y and Y¹ contains at least one hydroxyl substituent;

polyamides of formula (III) synthesized with at least one portion of an activated diacid (diacid chloride, dianhydride or diester) instead of the diacid;

polyamides of formula (III) in which X represents —(CH₂)₃— or —(CH₂)₁₀; and

polyamides of formula (III) in which the polyamides end with a monofunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

According to the invention, the end groups of the polymer chain may end with:

a C₁ to C₅₀ alkyl ester group by introducing a C₁ to C₅₀ monoalcohol during the synthesis,

a C₁ to C₅₀ alkylamide group by taking as stopping group a monoacid if the silicone is α,ω-diaminated, or a monoamine if the silicone is an α,ω-dicarboxylic acid.

According to one embodiment variant of the invention, it is possible to use a copolymer of silicone polyamide and of hydrocarbon-based polyamide, i.e. a copolymer comprising moieties of formula (III) or (IV) and hydrocarbon-based polyamide moieties. In this case, the polyamide-silicone moieties may be arranged at the ends of the hydrocarbon-based polyamide.

Polyamide-based structuring agents containing silicones may be produced by silylic amidation of polyamides based on fatty acid dimer. This approach involves the reaction of free acid sites existing on a polyamide as end sites, with organosiloxane-monoamines and/or organosiloxane-diamines (amidation reaction), or alternatively with oligosiloxane alcohols or oligosiloxane diols (esterification reaction). The esterification reaction requires the presence of acid catalysts, as is known in the art. It is desirable for the polyamide containing free acid sites, used for the amidation or esterification reaction, to have a relatively high number of acid end groups (for example polyamides with high acid numbers, for example from 15 to 20).

For the amidation of the free acid sites of the hydrocarbon-based polyamides, siloxane diamines with 1 to 300, more particularly 2 to 50 and better still 2, 6, 9.5, 12, 13.5, 23 or 31 siloxane groups, may be used for the reaction with hydrocarbon-based polyamides based on fatty acid dimers. Siloxane diamines containing 13.5 siloxane groups are preferred, and the best results are obtained with the siloxane diamine containing 13.5 siloxane groups and polyamides containing high numbers of carboxylic acid end groups.

The reactions may be carried out in xylene to extract the water produced from the solution by azeotropic distillation, or at higher temperatures (about 180 to 200° C.) without solvent. Typically, the efficacy of the amidation and the reaction rates decrease when the siloxane diamine is longer, that is to say when the number of siloxane groups is higher. Free amine sites may be blocked after the initial amidation reaction of the diaminosiloxanes by reacting them either with a siloxane acid, or with an organic acid such as benzoic acid.

For the esterification of the free acid sites on the polyamides, this may be performed in boiling xylene with about 1% by weight, relative to the total weight of the reagents, of para-toluenesulphonic acid as catalyst.

These reactions carried out on the carboxylic acid end groups of the polyamide lead to the incorporation of silicone moieties only at the ends of the polymer chain.

It is also possible to prepare a copolymer of polyamide-silicone, using a polyamide containing free amine groups, by amidation reaction with a siloxane containing an acid group.

It is also possible to prepare a structuring polymer based on a copolymer between a hydrocarbon-based polyamide and a silicone polyamide, by transamidation of a polyamide having, for example, an ethylene-diamine constituent, with an oligosiloxane-α,ω-diamine, at high temperature (for example 200 to 300° C.), to carry out a transamidation such that the ethylenediamine component of the original polyamide is replaced with the oligosiloxane diamine.

The copolymer of hydrocarbon-based polyamide and of polyamide-silicone may also be a grafted copolymer comprising a hydrocarbon-based polyamide backbone with pendent oligosiloxane groups.

This may be obtained, for example:

by hydrosilylation of unsaturated bonds in polyamides based on fatty acid dimers;

by silylation of the amide groups of a polyamide; or

by silylation of unsaturated polyamides by means of an oxidation, that is to say by oxidizing the unsaturated groups into alcohols or diols, to form hydroxyl groups that are reacted with siloxane carboxylic acids or siloxane alcohols. The olefinic sites of the unsaturated polyamides may also be epoxidized and the epoxy groups may then be reacted with siloxane amines or siloxane alcohols.

According to a second embodiment of the invention, the structuring polymer consists of a homopolymer or a copolymer comprising urethane or urea groups.

As previously, the polymer may comprise polyorganosiloxane moieties containing two or more urethane and/or urea groups, either in the backbone of the polymer or on side chains or as pendent groups.

The polymers comprising at least two urethane and/or urea groups in the backbone may be polymers comprising at least one moiety corresponding to the following formula:

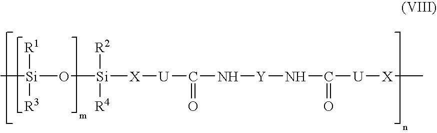

in which R¹, R², R³, R⁴, X, Y, m and n have the meanings given above for formula (I), and U represents —O— or —NH—, such that:

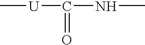

corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. Preferably, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to the formula:

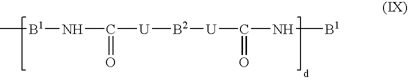

in which B¹ is a group chosen from the groups given above for Y, U is —O— or —NH— and B² is chosen from:

linear or branched C₁ to C₄₀ alkylene groups, which can optionally bear an ionizable group such as a carboxylic acid or sulphonic acid group, or a neutralizable or quaternizable tertiary amine group,

C₅ to C₁₂ cycloalkylene groups, optionally bearing alkyl substituents, for example one to three methyl or ethyl groups, or alkylene, for example the diol radical: cyclohexanedimethanol,

phenylene groups that may optionally bear C₁ to C₃ alkyl substituents, and

groups of formula:

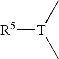

in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.

T can represent, for example:

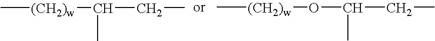

with w being an integer ranging from 1 to 10 and R⁵ being a polyorganosiloxane chain.

When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C, to C₄₀ alkylene group, in particular —(CH₂)₂— or —(CH₂)₆— or a group:

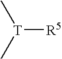

with R⁵ being a polyorganosiloxane chain.

As previously, the structuring polymer may be formed from silicone urethane and/or silicone urea moieties of different length and/or constitution, and may be in the form of block or random copolymers.

According to the invention, the silicone may also comprise urethane and/or urea groups no longer in the backbone but as side branches.

In this case, the polymer may comprise at least one moiety of formula:

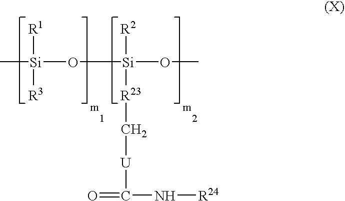

in which R¹, R², R³, m₁ and m₂ have the meanings given above for formula (I),

U represents O or NH,

R²³ represents a C₁ to C₄₀ alkylene group, optionally comprising one or more hetero atoms chosen from O and N, or a phenylene group, and

R²⁴ is chosen from linear, branched or cyclic, saturated or unsaturated C₁ to C₅₀ alkyl groups, and phenyl groups optionally substituted with one to three C₁ to C₃ alkyl groups.

The polymers comprising at least one moiety of formula (X) contain siloxane units and urea or urethane groups, and they may be used as structuring agents in the compositions of the invention.

The siloxane polymers may have a single urea or urethane group by branching or may have branches containing two urea or urethane groups, or alternatively they may contain a mixture of branches containing one urea or urethane group and branches containing two urea or urethane groups.

They may be obtained from branched polysiloxanes, comprising one or two amino groups by branching, by reacting these polysiloxanes with monoisocyanates.

As examples of starting polymers of this type containing amino and diamino branches, mention may be made of the polymers corresponding to the following formulae:

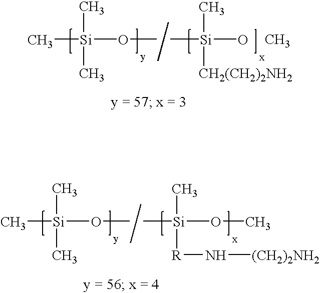

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branching may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polymers that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula:

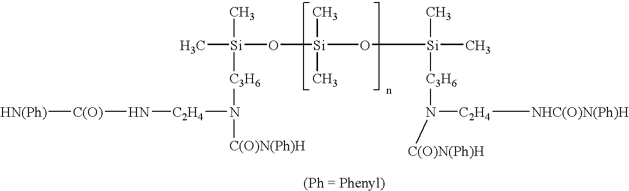

in which Ph is a phenyl group and n is a number from 0 to 300, in particular from 0 to 100, for example 50.

This polymer is obtained by reacting the following polysiloxane containing amino groups:

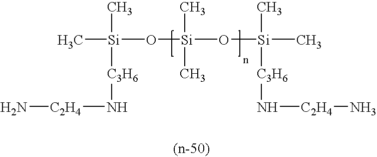

with phenyl isocyanate.

The polymers of formula (VIII) comprising urea or urethane groups in the chain of the silicone polymer may be obtained by reaction between a silicone containing α,ω-NH₂ or —OH end groups, of formula:

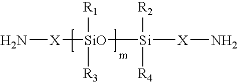

in which m, R¹, R², R³, R⁴ and X are as defined for formula (I) and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoichiometric proportions between the two reagents, diisocyanate and coupling agent, Y may have the formula (IX) with d equal to 0 or d equal to 1 to 5.

As in the case of the polyamide silicones of formula (II) or (III), it is possible to use in the invention polyurethane or polyurea silicones containing moieties of different length and structure, in particular moieties whose lengths differ by the number of silicone units. In this case, the copolymer may correspond, for example, to the formula:

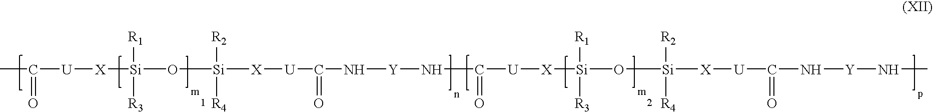

in which R¹, R², R³, R⁴, X, Y and U are as defined for formula (VIII) and m₁, m₂, n and p are as defined for formula (V).

Branched polyurethane or polyurea silicones may also be obtained using, instead of the diisocyanate OCN—Y—NCO, a triisocyanate of formula:

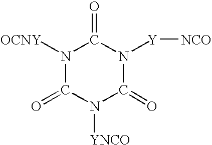

A polyurethane or polyurea silicone containing branches comprising an organosiloxane chain with groups capable of establishing hydrogen interactions is thus obtained. Such a polymer comprises, for example, a moiety corresponding to the formula:

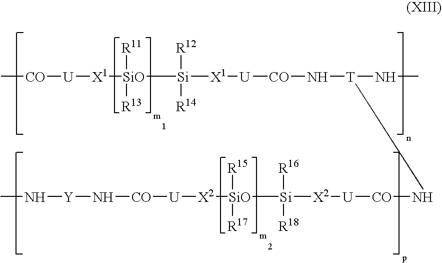

in which X¹ and X², which are identical or different, have the meaning given for X in formula (I), n is as defined in formula (I), Y and T are as defined in formula (I), R¹¹ to R¹⁸ are groups chosen from the same group as R¹ to R⁴, m₁ and m₂ are numbers in the range from 1 to 1 000, and p is an integer ranging from 2 to 500.

As in the case of the polyamides, this copolymer can also comprise polyurethane silicone moieties without branching.

In this second embodiment of the invention, the siloxane-based polyureas and polyurethanes that are preferred are:

polymers of formula (VIII) in which m is from 15 to 50;

mixtures of two or more polymers in which at least one polymer has a value of m in the range from 15 to 50 and at least one polymer has a value of m in the range from 30 to 50;

polymers of formula (XII) with m₁ chosen in the range from 15 to 50 and m₂ chosen in the range from 30 to 500 with the portion corresponding to m₁ representing 1% to 99% by weight of the total weight of the polymer and the portion corresponding to m₂ representing 1% to 99% by weight of the total weight of the polymer;

mixtures of polymer of formula (VIII) combining

1) 80% to 99% by weight of a polymer in which n is equal to 2 to 10 and in particular 3 to 6, and

2) 1% to 20% of a polymer in which n is in the range from 5 to 500 and in particular from 6 to 100,

copolymers comprising two moieties of formula (VIII) in which at least one of the groups Y contains at least one hydroxyl substituent;

polymers of formula (VIII) synthesized with at least one portion of an activated diacid (diacid chloride, dianhydride or diester) instead of the diacid;

polymers of formula (VIII) in which X represents —(CH₂)₃— or —(CH₂)₁₀—; and

polymers of formula (VIII) in which the polymers end with a multifunctional chain chosen from the group consisting of monofunctional amines, monofunctional acids, monofunctional alcohols, including fatty acids, fatty alcohols and fatty amines, such as, for example, octylamine, octanol, stearic acid and stearyl alcohol.

As in the case of the polyamides, copolymers of polyurethane or polyurea silicone and of hydrocarbon-based polyurethane or polyurea may be used in the invention by performing the reaction for synthesizing the polymer in the presence of an α,ω-difunctional block of non-silicone nature, for example a polyester, a polyether or a polyolefin.

As has been seen previously, the structuring polymer consisting of homopolymers or copolymers of the invention may contain siloxane moieties in the main chain of the polymer and groups capable of establishing hydrogen interactions, either in the main chain of the polymer or at the ends thereof, or on side chains or branches of the main chain. This may correspond to the following five arrangements:

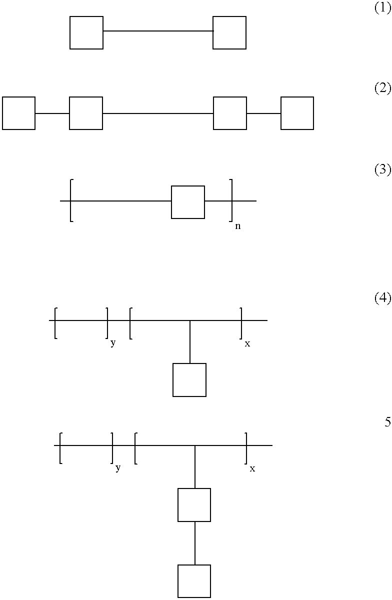

in which the continuous line is the main chain of the siloxane polymer and the squares represent the groups capable of establishing hydrogen interactions.

In case (1), the groups capable of establishing hydrogen interactions are arranged at the ends of the main chain.

In case (2), two groups capable of establishing hydrogen interactions are arranged at each of the ends of the main chain.

In case (3), the groups capable of establishing hydrogen interactions are arranged within the main chain in repeating moieties.

In cases (4) and (5), these are copolymers in which the groups capable of establishing hydrogen interactions are arranged on branches of the main chain of a first series of moieties that are copolymerized with moieties not comprising groups capable of establishing hydrogen interactions. The values n, x and y are such that the polymer has the desired properties in terms of an agent for gelling fatty phases based on silicone oil.

According to the invention, the structuring of the liquid fatty phase containing at least one silicone oil is obtained with the aid of one or more of the polymers mentioned above, in combination with solid particles with a hydrophobic surface.

As examples of polymers that may be used, mention may be made of the silicone polyamides obtained in accordance with Examples 1 and 2 of document U.S. Pat. No. 5,981,680.

The at least one structuring polymer in the compositions of the invention may have a softening point greater than 50° C., such as from 65° C. to 190° C., and for example less than 150° C., and further such as from 70° C. to 130° C., and even further such as from 80° C. to 105° C. This softening point may be lower than that of structuring polymers used in the art which may facilitate the use of the at least one structuring polymer of the present invention and may limit the degradation of the liquid fatty phase. These polymers may be non waxy polymers.

The softening point can be measured by a well known method as “Differential Scanning Calorimetry” (i.e. DSC method) with a temperature rise of 5 to 10° C./min.

The at least one structuring polymer has good solubility in the silicone oils and produces macroscopically homogeneous compositions. Preferably, they have an average molecular mass from 500 to 200,000, for example from 1,000 to 100,000 and preferably from 2,000 to 30,000.

Further, an embodiment of the invention relates to a skin, lips, or keratinous fibers care, treatment, or make-up composition comprising at least one liquid fatty phase comprising (i) at least one oil structured with at least one of the above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Additionally, an embodiment of the invention relates to a skin, lips, or keratinous fibers care or make-up composition comprising structured composition containing at least one liquid fatty phase comprising (i) at least one oil structured with at least one above mentioned structuring polymer (ii) at least one silicone gum, and at least one coloring agent, said oil having an affinity (compatibility) with said structuring polymer and/or with the silicone gum, and the liquid fatty phase, the polymer and the short chai ester forming a physiologically acceptable medium.

Another embodiment of the invention relates to a mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Another embodiment of the invention relates to a deodorant product or a care product for the skin or body comprising an anhydrous composition containing at least one liquid fatty phase comprising (i) at least one oil structured with at least one of the above mentioned structuring polymer and (ii) at least one silicone gum, said oil having an affinity (compatibility) with said structuring polymer and/or with the silicone gum, and the liquid fatty phase, the polymer and the silicone gum forming a physiologically acceptable medium.

Another embodiment of the invention relates to a lipstick composition comprising an anhydrous composition containing at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Another embodiment of the invention relates to a method for care, make-up or treatment of keratin materials comprising applying to the keratin materials an anhydrous composition containing at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Another embodiment of the invention relates to a method for care, make-up or treatment of keratinous fibers, lips, or skin comprising applying to the keratinous fibers, lips, or skin a composition comprising at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Another embodiment of the invention relates to a method for providing an anhydrous composition having at least one property chosen from non-exudation, gloss, and comfortable deposit on keratin materials chosen from lips, skin, and keratinous fibers, comprising including in the composition at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Another embodiment of the invention relates to a method of making up or caring for skin, lips or keratinous fibers comprising applying to the skin, lips, or keratinous fibers a structured composition containing at least one liquid fatty phase comprising (i) at least one oil structured with at least one above mentioned structuring polymer and (ii) at least one silicone gum, said oil having an affinity with said structuring polymer and/or with the silicone gum, and the liquid fatty phase, the polymer and the silicone gum forming a physiologically acceptable medium.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 900 gf, and further such as from 20 gf to 600 gf.

This hardness is measured in one of two ways. A first test for hardness is according to a method of penetrating a probe into the composition and in particular using a texture analyzer (for example TA-XT2 from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, and further such as from 30 gf to 200 gf.

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

As is evident, the hardness of the composition according to the invention may, for example, be such that the composition is advantageously self-supporting and can disintegrate easily to form a satisfactory deposit on the skin and/or the lips and/or superficial body growths, such as keratinous fibers. In addition, with this hardness, the composition of the invention may have good impact strength.

According to the invention, the composition in stick form may have the behavior of a deformable, flexible elastic solid, giving noteworthy elastic softness on application. The compositions in stick form of the prior art do not have these properties of elasticity and flexibility.

Liquid Fatty Phase

For the purposes of the invention, the expression “liquid fatty phase” means a fatty phase which is liquid at room temperature (25° C.) and atmospheric pressure (760 mmHg, i.e. 101 kPa), composed of one or more fatty substances that are liquid at room temperature, also referred to as oils, that are generally mutually compatible, i.e. forming a homogeneous phase macroscopically. The expression “liquid fatty substance” means a non-aqueous liquid medium which is immiscible in all proportions with water, for example, a hydrocarbon-based compound comprising one or more carbon chains each containing at least 5 carbon atoms and possibly comprising at least one polar group chosen from carboxylic acid, hydroxyl, polyol, amine, amide, phosphoric acid, phosphate, ester, ether, urea, carbamate, thiol, thioether and thioester, a silicone compound optionally comprising carbon chains at the end or pendant, these chains optionally being substituted with a group chosen from fluoro, perfluoro, (poly)amino acid, ether, hydroxyl, amine, acid and ester groups; or a fluoro or perfluoro compound such as fluorohydrocarbons or perfluorohydrocarbons containing at least 5 carbon atoms, possibly comprising a hetero atom chosen from N, O, S and P and optionally at least one function chosen from ether, ester, amine, acid, carbamate, urea, thiol and hydroxyl groups.

The at least one liquid, in one embodiment, may comprise at least one oil having an affinity with the structuring polymer and/or with the silicone gum. The at least one oil, for example, may be chosen from polar oils and apolar oils including hydrocarbon-based liquid oils and oily liquids at room temperature. In one embodiment, the composition of the invention comprises at least one structuring polymer, at least one silicone gum and at least one apolar oil. The apolar oils of the invention, for example, may be added to a polar oil, the apolar oils acting in particular as co-solvent for the polar oils.

The liquid fatty phase of the composition may contain more than 30%, for example, more than 40%, of liquid oil(s) containing a group similar to that of the units of the structuring polymer, and for example from 50% to 100%. In one embodiment, the liquid fatty phase structured with a silicone-polyamide-type skeleton contains a high quantity, i.e., greater than 30%, for example greater than 40% relative to the total weight of the liquid fatty phase, or from 50% to 100%, of at least one apolar, such as hydrocarbon-based oil, silicone oils or mixtures thereof. For the purposes of the invention, the expression “hydrocarbon-based oil” means an oil essentially comprising carbon and hydrogen atoms, optionally with at least one group chosen from hydroxyl, ester, carboxyl and ether groups.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase.

For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms, phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof.

In one embodiment, the liquid fatty phase comprises one or more silicone oils, in particular phenylsilicones such as phenyl trimethicones.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase can be, for example, present in an amount ranging from 1% to 99% by weight relative to the total weight of the composition, for example from 5% to 99%, 5% to 95.5%, from 10% to 80% or from 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg (1.33 to 40 000 Pa) and, for example, greater than 0.03 mmHg (4 Pa) and further example greater than 0.3 mmHg (40 Pa). The expression “non-volatile oil” means an oil which remains on the skin or the lips at room temperature and atmospheric pressure for at least several hours, such as those having a vapor pressure of less than 10⁻² mmHg (1.33 Pa).

According to the invention, these volatile solvents may facilitate the staying power or long wearing properties of the composition on the skin, the lips or superficial body growths such as nails and keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, can be present in an amount ranging from 0% to 95.5% relative to the total weight of the composition, such as from 2% to 75% or, for example, from 10% to 45%. This amount will be adapted by a person skilled in the art according to the desired staying power or long wearing properties.

The at least one liquid fatty phase of the composition of the invention may further comprises a dispersion of lipid vesicles. The composition of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles, or a fatty phase optionally containing dispersed lipid vesicles. In one embodiment, the composition has a continuous oily phase or fatty phase and is more specifically an anhydrous composition in, for example, a stick or dish form.

An anhydrous composition is one that has less than 10% water by weight, such as, for example, less than 5% by weight.

The Silicone Gums

For the purpose of the invention, silicone gums means a polymeric silicone with repeating units of siloxane, having a viscosity greater than 2,500 cSt. These silicones are also known as polydiorganosiloxanes and correspond to the following formula:

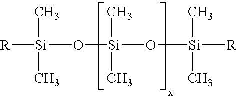

In this formula the terminal Si's can also be other than methyl and may be represented with substitutions on the repeating Si such that the R group is an alkyl of 1 to 6 carbon atoms ALKYL OF 1 TO 6 CARBONS, which may be linear, branched and/or functionalized selected from methyl, ethyl, propyl, isopropyl, butyl, isobutyl, t-butyl, amyl, hexyl, vinyl, allyl, cycohexyl, phenyl, fluoroalkyl, and mixtures thereof. The silicone gums employed in the present invention may be terminated by triorganosilyl groups of the formula R′₃ where R′ is a radical of monovalent hydrocarbons containing from 1 to 6 carbon atoms, hydroxyl groups, alkoxyl groups and mixtures thereof. The silicone gums used in these invention have an affinity with the structuring polymer and/or with the silicone gum, and the liquid fatty phase, the polymer and the silicone gum form a physiologically acceptable medium.

A particularly preferred fluid diorganopolysiloxane polymer is poly(dimethylsiloxane), herein referred to as PDMS. Also useful is a mixture of silicone gums such as the commaercially available DC 1503 which is a blend of dimethicone and dimethiconol. Other useful silicone gums are DC 1428 fluid (Dow Corning) and those silicone gums described in U.S. Pat. No. 4,574,082, the contents of which are incorporated herein by reference.

Additional Additives

Short Chain Esters

According to the invention, the esters may either be monoesters, diesters or polyesters. These esters may be linear, branched or cyclic, saturated or unsaturated. These esters should preferably be branched and saturated. They may also be aliphatic or aromatic. These esters may have from 6 to 25 carbon atoms and particularly from 14 to 22 carbon atoms. They may be chosen amongst acid esters having from 2 to 18 carbon atoms, and particularly amongst alcohol esters having from 2 to 20 carbon atoms or amongst polyols having from 2 to 8 carbon atoms or their mixtures, on condition that the number of carbon atoms is higher than 10, so that the ester is not volatile and penetrates the skin.

Particularly, these esters are hydrocarbon-based esters which correspond to the following formula RCOOR′ where R represents a residue of fatty acid having from 1 to 29 carbon atoms, and R′ represents a hydrocarbon-based chain containing from 2 to 30 carbon atoms, on condition that the number of carbon atoms in R′ is higher than 10, so that the ester is not volatile and penetrates the skin.

The ester may be chosen among a non-limitative list including the following:

Neopentanoic acid esters such as isodecyl neopentanoate, isotridecyl neopentanoate, isostearyl neopentanoate, octyldocecyl neopentanoate,

Isononanoic acid esters such as isononyl isononanoate, octyl isononanoate, isodecyl isononanoate, isotridecyl isononanoate, isostearyl isononanoate, ethylhexyl isononanoate

Isopropylic alcohol esters, such as isopropyl myristate, isopropyl palmitate, isopropyl stearate or isostearate, isopropyl laurate, diisopropyl adipate

Alkyl or polyalkyl octanoates, decanoates or ricinoleates, such as cetyl octanoate, tridecyl octanoate

Polyalkylene glycol esters, such as polyethylene glycol diheptanoate, hexanoate-2-diethyl propylene glycol and their mixtures

Benzoate alkyls particularly benzoate alkyls having from 12 to 15 carbon atoms

Hydroxylated esters such as isotearyl lactate and diisostearyl malate

Pentaerythritol esters.

Examples of short chain esters also include purcellin oil (cetostearyl octanoate), ethylhexyl ethylhexanoate, dicapryl ester, 2-ethylhexyl palmitate, 2-ethyl-palmitate and isostearyl isostearate.

The isononyl isononanoate and diisostearyl malate are particularly suited for the embodiment of this invention.

This or these hydrocarbon-based ester(s) may be used in the composition at a percentage of 5 to 90%, notably of 10 to 60%, particularly of 20 to 50% by weight of the total weight of the composition.

The mass ratio between the short chain ester and the structuring polymer is preferably between 1/4 and 2/1, more preferably between 1/3 and 1/1.

Additional ingredients which offer similar cosmetic properties as the short chain esters are short chain ethers which may be represented as

J-O—K

where J and K are identical or different and represent a linear or branched alkyl radical from 1 to 40 carbon atoms, preferably from 7 to 19 carbon atoms, possibly including one or more double bonds. An example of such an ether includes dicapryl ether.

Crystalline Silicone Compounds

Additional ingredients which may be used in the composition include crystalline silicone compounds. A crystalline silicone compound is a compound comprising silicone in its molecule, which is solid at room temperature, and has a crystalline character. This compound or class of compounds is compatible with the liquid fatty phase and the structuring agent.

The crystalline silicone compounds belong to a class of alkyl siloxane waxes corresponding to the formulae below: 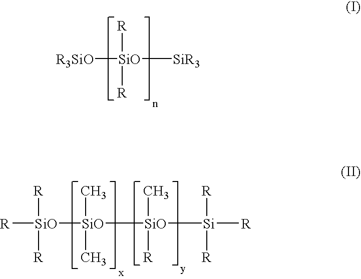 [(CH₃)₃SiO]₂(CH₃)SiR

(CH₃)₃SiO[(CH₃)₂SiO]ₓ(RCH₃SiO)_{y}Si(CH₃)₃

This could also be written as R₃SiO[(CH₃)₂SiO]ₓ(RCH₃SiO)_{y}SiR₃

where R is an alkyl chain. x may be 0. The substituent R may be as low as 1 or as high as 50 or more as long as this silicone compound crystallizes at room temperature.

Examples of crystalline silicone compounds include, but are not limited to, C20-24 Alkyl Methicone, C24-28 Alkyl Dimethicone, C20-24 Alkyl Dimethicone, C24-28 Alkyl Dimethicone commercially available from Archimica Fine Chemicals, Gainesville, Fla. under the designation of SilCare 41M40, SilCare 41M50, SilCare 41M70 and SilCare 41M80. Stearyl Dimethicone available as SilCare 41M65 from Archimica or as DC-2503 from Dow-Corning, Midland, Mich. Similarly, stearoxytrimethylsilane sold as SilCare 1M71 or DC-580 may be used in an embodiment of this invention. Furthermore, similar crystalline compounds are available from Degussa Care Specialties, Hopewell, Va. under the designation ABIL Wax 9810, 9800, or 2440, or Wacker-Chemie GmbH, Burghausen, Germany, under the designation BelSil SDM 5055, or OSi Specialties, Greenwich, Conn. under the designation Silsoft. Other crystalline silicone compounds include C30-45 Alkyl Methicone available from Dow Corning as AMS-C30 Wax, as well as GE's SF1642, or SF-1632 available from General Electric, Fairfield, Conn.

Additional Additives

The composition of the invention can also comprise any additive usually used in the field under consideration, chosen in particular from dispersants such as poly(2-hydroxystearic acid), antioxidants, essential oils, preserving agents, fragrances, waxes, liposoluble polymers that are dispersible in the medium, fillers, neutralizing agents, cosmetic and dermatological active agents such as, for example, emollients, moisturizers, vitamins, essential fatty acids, sunscreens, and mixtures thereof. These additives may be present in the composition in a proportion of from 0% to 20% (such as from 0.01% to 20%) relative to the total weight of the composition and further such as from 0.01% to 10% (if present).

The composition of the invention can also contain, as an additive, an aqueous phase containing water that is optionally thickened or gelled with an aqueous-phase thickener or gelling agent and/or containing ingredients soluble in water. The water can represents from 0.01 to 50%, for example from 0.5 to 30% relative to the total weight of the composition.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted or non tinted dermatological composition or a care composition for keratin materials such as the skin, the lips and/or superficial body growths, in the form of an antisun composition or body hygiene composition in particular in the form of a deodorant product or make-up-removing product in stick form. It can be used in particular as a care base for the skin, superficial body growths or the lips (lip balms, for protecting the lips against cold and/or sunlight and/or the wind, or care cream for the skin, the nails or the hair). As defined herein, a deodorant product is personal hygiene product and does not relate to care, make-up or treatment of keratin materials, including keratinous fibers.

The composition of the invention may also be in the form of a colored make-up product for the skin, in particular a foundation, optionally having care or treating properties, a blusher, a face powder, an eye shadow, a concealer product, an eyeliner, a make-up product for the body; a make-up product for the lips such as a lipstick, optionally having care or treating properties; a make-up product for superficial body growths such as the nails or the eyelashes, in particular in the form of a mascara cake, or for the eyebrows and the hair, in particular in the form of a pencil.

Needless to say, the composition of the invention should be cosmetically or dermatologically acceptable, i.e. it should contain a non-toxic physiologically acceptable medium and should be able to be applied to the skin, superficial body growths or the lips of human beings. For the purposes of the invention, the expression “cosmetically acceptable” means a composition of pleasant appearance, odor, feel and taste.

The composition advantageously contains at least one cosmetic active agent and/or at least one dermatological active agent, i.e., an agent having a beneficial effect on the skin, lips or body growths and/or at least one coloring agent.

Coloring Agents

The composition may further comprise at least one coloring agent. The coloring agent according to the invention may be chosen from the lipophilic dyes, hydrophilic dyes, pigments and nacreous pigments (i.e., nacres) usually used in cosmetic or dermatological compositions, and mixtures thereof. This coloring agent is generally present in a proportion of from 0.01% to 50% relative to the total weight of the composition, such as from 0.5% to 40% and further such as from 5% to 30%, if it is present. In the case of a composition in the form of a free or compacted powder, the amount of coloring agent in the form of solid particles that are insoluble in the medium (nacres and/or pigments) may be up to 90% relative to the total weight of the composition.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow or annatto. They can represent from 0.1% to 20% of the weight of the composition, for example, from 0.1% to 6% (if present). The water-soluble dyes are, for example, beetroot juice or methylene blue, and can represent up to 6% of the total weight of the composition.

The pigments may be white or colored, goniochromatic or not, mineral and/or organic, and coated or uncoated. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments which may be mentioned are carbon black, pigments of D & C type, and lakes based on cochineal carmine or on barium, strontium, calcium or aluminum. The pigments can represent from 0.1% to 50%, such as from 0.5% to 40% and further such as from 2% to 30% relative to the total weight of the composition, if they are present.

The nacreous pigments may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the type mentioned above, as well as nacreous pigments based on bismuth oxychloride. They can represent, for example, from 0.1% to 20% relative to the total weight of the composition, and further such as from 0.1% to 15%, if they are present.

In one embodiment, the coloring agent is a pigment (nacreous or not).

Waxes

The composition can optionally contain one or more waxes to improve the structuring in stick form, although this rigid form can be obtained in the absence of wax. For the purposes of the present invention, a wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C., and having an anisotropic crystal organization in the solid state. The size of the crystals is such that the crystals diffract and/or scatter light, giving the composition a cloudy, more or less opaque appearance. By bringing the wax to its melting point, it is possible to make it miscible with oils and to form a microscopically homogeneous mixture, but on returning the temperature of the mixture to room temperature, recrystallization of the wax in the oils of the mixture is obtained. It is this recrystallization in the mixture which is responsible for the reduction in the gloss of the mixture. Thus, the composition advantageously contains little or no wax, and in particular less than 5% wax.

For the purposes of the invention, the waxes are those generally used in cosmetics and dermatology; they are, for example, of natural origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C.

According to the invention, the melting point values correspond to the melting peak measured by the “Differential Scanning Calorimetry” method with a temperature rise of 5 or 10° C./min.

Liposoluble or Dispersible Polymers

The composition of the invention also can contain at least one polymer that is liposoluble or dispersible in the medium, other than the structuring polymer and the at least one rheological agent, and may have film-forming properties and may have, for example, an average molecular weight of from 500 to 1,000,000, such as from 1,000 to 500,000, and for example, further such as from 5,000 to 100,000, and even further such as from 5,000 to 20,000. This at least one liposoluble polymer may contribute towards increasing the viscosity and/or improving the staying power of the film. The at least one liposoluble polymer can have a softening point of not more than 30° C.

As examples of liposoluble polymers which can be used in the invention, mention may be made of: polyalkylenes, in particular polybutene, poly(meth)acrylates, alkylcelluloses with a linear or branched, saturated or unsaturated C₁ to C₈ alkyl radical, such as ethylcellulose and propylcellulose, silicone polymers that are compatible with the fatty phase, as well as vinylpyrrolidone (VP) copolymers, and mixtures thereof.

Vinylpyrrolidone copolymers, copolymers of a C₂ to C₃₀, such as C₃ to C₂₂ alkene, and combinations thereof, can be used. As examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

Not only for the staying power properties but also for the feel and consistency properties of the film, the PVP/hexadecene copolymer having an average molecular weight of from 7,000 to 7,500 or alternatively the PVP/eicosene copolymer having an average molecular weight of from 8,000 to 9,000 can be used.

The liposoluble or dispersible polymers in the composition of the invention can be also used in an amount of from 0.01% to 20% (as active material) relative to the total weight of the composition, such as, for example, from 1% to 10%, if they are present.

The composition according to the invention may be manufactured by the known processes that are generally used in cosmetics or dermatology. It may be manufactured by the process which comprises heating the polymer at least to its softening point, adding the silicone gum, the coloring agent(s) and the additive(s) thereto and then mixing everything together until a clear, transparent solution is obtained. After reducing the temperature, the volatile solvent(s) is(are) then added to the mixture obtained. The homogeneous mixture obtained can then be cast in a suitable mould such as a lipstick mould or directly into the packaging articles (case or dish in particular).

Another aspect of the invention is a lipstick composition in stick form at least one liquid fatty phase comprising (i) at least one oil structured with at least one structuring polymer consisting of a polymer (homopolymer or copolymer) with a weight-average molecular mass ranging from 500 to 500,000, containing at least one moiety comprising:

at least one polyorganosiloxane group, consisting of from 1 to 1,000 organosiloxane units in the chain of the moiety or in the form of a graft, and

at least two groups capable of establishing hydrogen interactions, chosen from ester, amide, sulphonamide, carbamate, thiocarbamate, urea, urethane, thiourea, oxamido, guanamido and biguanidino groups, and combinations thereof,

the polymer being solid at room temperature and soluble in the liquid fatty phase at a temperature of from 25 to 250° C., and

(ii) at least one silicone gum, said oil having an affinity with said structuring polymer and/or with the silicone gum, and the liquid fatty phase, the polymer and the silicone gum forming a physiologically acceptable medium.

The crystalline silicone compound and the structuring polymer can give the composition the appearance of a deformable elastic solid with a hardness ranging from 30 to 300 gf, such as 30 to 250 gf, and further such as 30 to 200 gf, even in the absence of wax. The hardness is measured by the “cheese-wire” method described above. The non-waxy polymer may be a polymer whose skeleton comprises units containing a hetero atom, as defined previously, and further may be a polyamide that may contain (an) alkyl end group(s) linked to the skeleton via an ester group.

An aspect of the invention is also a care, make-up or treatment cosmetic process for keratin materials of human beings, and in particular the skin, the lips and superficial body growths, comprising the application to the keratin materials of the composition, in particular the cosmetic composition, as defined above.

An aspect of the invention is also a combination (i) of at least one polymer consisting of a polymer (homopolymer or copolymer) with a weight-average molecular mass ranging from 500 to 500,000, containing at least one moiety comprising:

at least one polyorganosiloxane group, consisting of from 1 to 1,000 organosiloxane units in the chain of the moiety or in the form of a graft, and

at least two groups capable of establishing hydrogen interactions, chosen from ester, amide, sulphonamide, carbamate, thiocarbamate, urea, urethane, thiourea, the polymer being solid at room temperature and soluble in the liquid fatty phase at a temperature of from 25 to 250° C., and

(ii) at least one silicone gum, said oil having an affinity with said structuring polymer and/or with the silicone gum, and the liquid fatty phase, the polymer and the silicone gum forming a physiologically acceptable medium, in a cosmetic composition or for the manufacture of a physiologically acceptable composition, to obtain a solid composition, such as a wax-free composition, which does not exude and/or which can produce a glossy and/or comfortable deposit on keratin materials.

The compositions of the present invention may also further comprise water, optionally thickened with an aqueous-phase thickener or gelled with a gelling agent and/or containing ingredients soluble in water.

The invention is illustrated in greater detail in the examples which follow. The amounts are given as percentages by mass.

The preparation procedures for the following examples are as such:

Composition without Pigments:

1. Mix Phase A until uniform and heat to 110° C.;

2. When the temperature of the oil bath reaches 110° C., add phase B with agitation until melted and the phase is uniform;

3. Mix the mass and let cool to 90°-95° C.;

4. Pour the bulk into lipstick molds.

Composition with Pigments:

1. Charge a portion of Phase A (the oil phase) into a Disconti Mill;

2. Heat to about 65° C. to 70° C.;

3. Add the pigments (Phase C). Mill for 40-45 minutes at 65° C. to 70° C. Check the dispersion for the absence of clumps. This forms the color phase;

4. Melt the wax and polymer structuring agent (Phase B) by heating to 105° C.-110° C. in a melting kettle;

5. Discharge the color phase from the mill

6. Rinse the mill with the remaining oil phase for 20-30 minutes;

7. Complete the color phase with the rinse residual;

8. Add the color phase into the melting kettle and heat to 103° C.-105° C.;

9. Mix for 20-30 minutes until homogeneous;

10. Let cool to 90° C.-95° C. and pour the bulk into lipstick molds.

### EXAMPLES

| | | |
|---|---|---|
| Ingredient INCI Name | Ingredient Trade Name | % w/w |

Example 1: Lip composition
Phase A

| | | |
|---|---|---|
| Hydrogenated Polyisobutene | Polysynlane V | 10.00 |
| Polygyceryl-2 Diisostearate | Dermol DGDIS | 10.00 |
| Diisostearyl Malate | Schercemol DISM | 16.00 |
| Phenyltrimethicone | DC 556 | 10.00 |
| Isononyl isononanoate | Wickenol 151 | 19.95 |
| Dimethicone and | DC 1503 Fluid | 10.00 |
| Dimethiconol | | |
| Phase B | | |
| Polyamidodimethylsiloxane | Example 3 of | 16.00 |
| | U.S. Pat. No. 5,981,680 | |
| Phase C | | |
| Pigments | Pigments | 3.05 |
| Mica | Mica Micro C-3000 | 2.00 |
| Silica | MSS-500/3H | 1.00 |
| Mica and Titanium Dioxide | Timiron Super Silver | 2.00 |

| | | |
|---|---|---|
| Ingredient INCI Name | Ingredient Trade Name | % w/w |

Example 2: Lip composition
Phase A

| | | |
|---|---|---|
| Polyglyceryl-2 diisostearate | Dermol DGDIS | 31.00 |
| Dimethicone | Dow Corning 200, 5 cSt | 14.89 |
| Dimethicone and | DC 1403 | 5.00 |
| dimethiconol | | |
| C12-15 Alkyl Benzoate | Finsolv TN | 9.00 |
| Isododecyl neopentanoate | DUB VCI 10 | 10.00 |
| Phase B | | |
| C30-45 alkyl dimethicone | SF-1642 | 10.00 |
| Polyamidodimethylsiloxane | Example 3 of | 15.00 |
| | U.S. Pat. No. 5,981,680 | |
| Phase C | | |
| Pigments | | 5.11 |
| Example 3: Lip balm | | |
| Phase A | | |
| Polyglyceryl-2 diisostearate | Dermol DGDIS | 43.0 |
| Dimethicone | Dow Corning 200, 5 cSt | 30.0 |
| C12-15 Alkyl Benzoate | Finsolv TN | 9.0 |
| Dimethicone | DC 1428 Fluid | 4.0 |
| Phase B | | |
| Stearyl Dimethicone | DC 2503 Cosmetic Wax | 4.0 |
| Polyamidodimethylsiloxane | Example 3 of | 10.0 |
| | U.S. Pat. No. 5,981,680 | |

The composition is hydrating, offers good shine, is comfortable to wear and has a silky feel.

The disclosures of U.S. Ser. No. 10/166,760 filed on Jun. 10, 2002 and U.S. Ser. No. 10/166,755 are incorporated herein by reference.

1. Composition comprising at least one liquid fatty phase comprising
(i) at least one oil structured with at least one structuring polymer consisting of a polymer (homopolymer or copolymer) with a weight-average molecular mass ranging from 500 to 500,000, containing at least one moiety comprising:
at least one polyorganosiloxane group, consisting of from 1 to 1,000 organosiloxane units in the chain of the moiety or in the form of a graft, and
at least two groups capable of establishing hydrogen interactions, chosen from ester, amide, sulphonamide, carbamate, thiocarbamate, urea, urethane, thiourea, oxamido, guanamido and biguanidino groups, and combinations thereof, the polymer being solid at 25° C. and soluble in the liquid fatty phase at a temperature of from 25 to 250° C., and
(ii) at least one silicone gum,
said oil having an affinity with said structuring polymer and/or said silicone gum, and wherein the liquid fatty phase, the structuring polymer and the silicone gum form a physiologically acceptable medium.

2. Composition according to claim 1, in which the structuring polymer comprises at least one moiety corresponding to the formula: 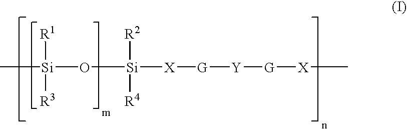
in which:
1) R¹, R², R³ and R⁴, which may be identical or different, represent a group chosen from:
linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,
C₆ to C₁₀ aryl groups, optionally substituted with one or more C, to C₄ alkyl groups,
polyorganosiloxane chains possibly containing one or more oxygen, sulphur and/or nitrogen atoms;
2) the groups X, which may be identical or different, represent a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulphur and/or nitrogen atoms, and/or bearing as substituent one of the following atoms or groups of atoms:
fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
4) Y represents a group corresponding to the formula: 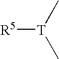
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁵ represents a linear or branched C₁ to C₅₀ alkyl group or a polyorganosiloxane chain, possibly comprising one or more ester, amide, urethane, thiocarbamate, urea, urethane, thiourea and/or sulphonamide groups, which may possibly be linked to another chain of the polymer;
5) The groups G, which may be identical or different, represent divalent groups chosen from: 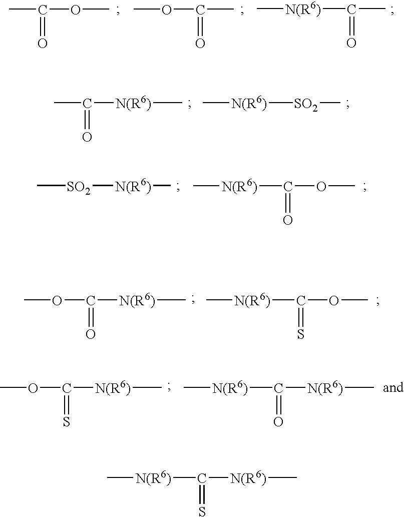
in which R⁶ represents a hydrogen atom or a linear or branched C₁ to C₂₀ alkyl group, on condition that at least 50% of the groups R⁶ of the polymer represents a hydrogen atom and that at least two of the groups G of the polymer are a group other than: 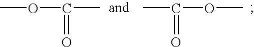
6) n is an integer ranging from 2 to 500 and preferably from 2 to 200, and m is an integer ranging from 1 to 1 000, preferably from 1 to 700 and better still from 6 to 200.

3. Composition according to claim 2, in which Y represents a group chosen from:
a) linear C₁ to C₂₀ and preferably C₁ to C₁₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,
g) polyorganosiloxane chains of formula: 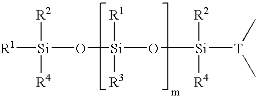
in which R¹, R², R³, R⁴, T and m are as defined above,
h) polyorganosiloxane chains of formula: 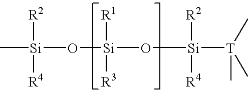
in which R¹, R², R³, R⁴, T and m are as defined above.

4. Composition according to claim 2, in which the structuring polymer comprises at least one moiety corresponding to formula (II): 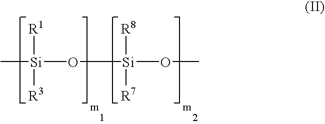
in which
R¹ and R³, which may be identical or different, are as defined above for formula (I),
R⁷ represents a group as defined above for R¹ and R³, or represents a group of formula —X-G-R⁹ in which X and G are as defined above for formula (I), and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R⁸ represents a group of formula —X-G-R⁹ in which X, G and R⁹ are as defined above,
m₁ is an integer ranging from 1 to 998, and
m₂ is an integer ranging from 2 to 500.

5. Composition according to claim 2, in which the polymer comprises at least one moiety of formula (ill) or (IV): 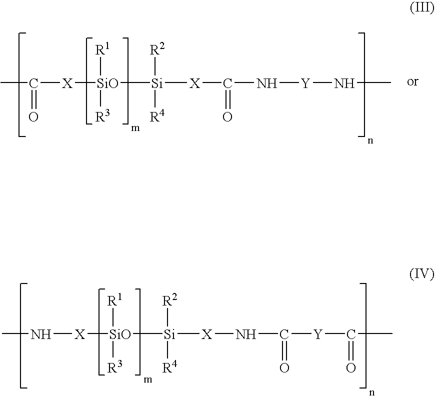
in which R¹, R², R³, R⁴, X, Y, m and n are as defined in claim 2.

6. Composition according to claim 2, in which X and/or Y represent an alkylene group containing in its alkylene portion at least one of the following elements:
1°) 1 to 5 amide, urea or carbamate groups,
2°) a C₅ or C₆ cycloalkyl group, and
3°) a phenylene group optionally substituted with 1 to 3 identical or different C₁ to C₃ alkyl groups, and/or substituted with at least one element chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

7. Composition according to claim 2, in which Y represents: 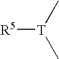
in which R⁵ represents a polyorganosiloxane chain and T represents a group of formula: 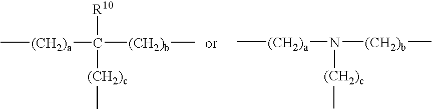
in which a, b and c are, independently, integers ranging from 1 to 10, and R¹⁰ is a hydrogen atom or a group such as those defined for R¹, R², R³ and R⁴, in claim 2.

8. Composition according to claim 2, in which R¹, R², R³ and R⁴ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

9. Composition according to claim 2, in which the structuring polymer comprises at least one moiety of formula: 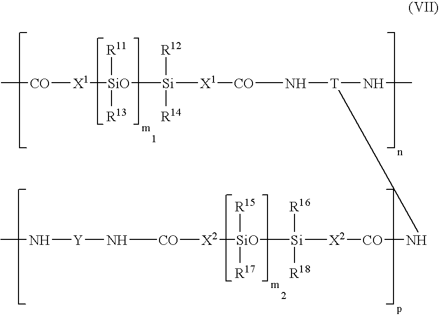
in which X¹ and X², which may be identical or different, have the meaning given for X in claim 2, n, Y and T are as defined in claim 2, R¹¹ to R¹⁸ are groups chosen from the same group as R¹ to R⁴ of claim 2, m₁ and m₂ are numbers in the range from 1 to 1,000, and p is an integer ranging from 2 to 500.

10. Composition according to claim 9, in which:
p is in the range from 1 to 25 and better still from 1 to 7,
R¹¹ to R¹⁸ are methyl groups,
T corresponds to one of the following formulae: 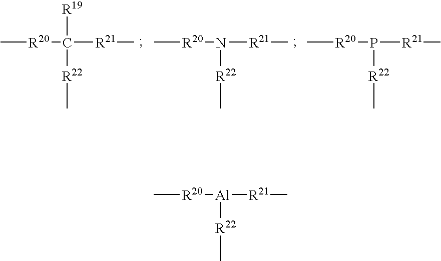
in which R¹⁹ is a hydrogen atom or a group chosen from the groups defined for R¹ to R⁴, and R²⁰, R²¹ and R²² are, independently, linear or branched alkylene groups, and more preferably corresponds to the formula: 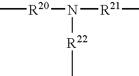
in particular with R²⁰, R²¹ and R²² representing —CH₂—CH₂—,
m₁ and m₂ are in the range from 15 to 500 and better still from 15 to 45,
X¹ and X² represent —(CH₂)₁₀—, and
Y represents —CH₂—.

11. Composition according to claim 2, in which the polymer comprises at least one moiety corresponding to the following formula: 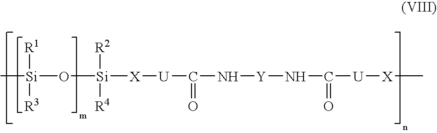
in which R¹, R², R³, R⁴, X, Y, m and n have the meanings given above for formula (I) in claim 2, and U represents —O— or —NH—, such that: 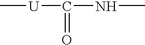
corresponds to a urethane or urea group,
or
Y represents a C₅ to C₁₂ cycloaliphatic or aromatic group that may be substituted with a C₁ to C₁₅alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane
or
Y represents a linear or branched C₁ to C₄₀ alkylene radical or a C₄ to C₁₂ cycloalkylene radical,
or
Y represents a polyurethane or polyurea block corresponding to the condensation of several diisocyanate molecules with one or more coupling agents of the diol or diamine type, corresponding to the formula: 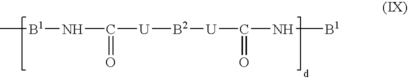
in which B¹ is a group chosen from the groups given above for Y, U is —O— or —NH— and B² is chosen from:
linear or branched C₁ to C₄₀ alkylene groups, which can optionally bear an ionizable group such as a carboxylic acid or sulphonic acid group, or a neutralizable or quaternizable tertiary amine group,
C₅ to C₁₂ cycloalkylene groups, optionally bearing alkyl substituents, for example one to three methyl or ethyl groups, or alkylene, for example the diol radical: cyclohexanedimethanol,
phenylene groups that may optionally bear C₁ to C₃ alkyl substituents, and groups of formula: 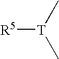
in which T is a hydrocarbon-based trivalent radical possibly containing one or more hetero atoms such as oxygen, sulphur and nitrogen and R⁵ is a polyorganosiloxane chain or a linear or branched C₁ to C₅₀ alkyl chain.

12. Composition according to claim 1, in which the polymer comprises at least one moiety of formula: 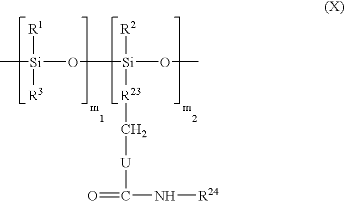
in which R¹, R², R³, m₁ and m₂ have the meanings given above for formula (I),
U represents O or NH,
R²³ represents a C₁ to C₄₀ alkylene group, optionally comprising one or more hetero atoms chosen from O and N, or a phenylene group, and
R²⁴ is chosen from linear, branched or cyclic, saturated or unsaturated C₁ to C₅₀ alkyl groups, and phenyl groups optionally substituted with one to three C₁ to C₃ alkyl groups.

13. Composition according to claim 10, in which the structuring polymer comprises at least one moiety of formula: 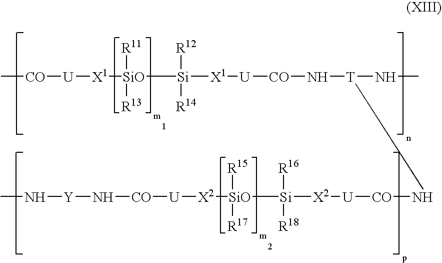
in which X¹ and X², which are identical or different, have the meaning given for X in claim 10, n, Y and T are as defined in claim 10, R¹¹ to R¹⁸ are groups chosen from the same group as R¹ to R⁴ of claim 10, m₁ and m₂ are numbers in the range from 1 to 1,000, and p is an integer ranging from 2 to 500.

14. Composition according to claim 1, in which the structuring polymer furthermore comprises a hydrocarbon-based moiety comprising two groups capable of establishing hydrogen interactions, chosen from ester, amide, sulphonamide, carbamate, thiocarbamate, urea, urethane, thiourea, oxamido, guanamido and biguanidino groups, and combinations thereof.

15. Composition according to claim 2, in which the structuring polymer furthermore comprises a hydrocarbon-based moiety comprising two groups capable of establishing hydrogen interactions, chosen from ester, amide, sulphonamide, carbamate, thiocarbamate, urea, urethane, thiourea, oxamido, guanamido and biguanidino groups, and combinations thereof.

16. Composition according to claim 1, in which the at least one structuring polymer represents from 0.5% to 80%, preferably from 2% to 60% and better still from 5% to 40%, relative to the total weight of the composition.

17. Composition according to claim 1, wherein said at least one structuring polymer has a softening point greater than 50° C.

18. Composition according to claim 1, wherein said at least one structuring polymer has a softening point of less than 150° C.

19. Composition according to claim 1, wherein said at least one structuring polymer has a softening point ranging from 70° C. to 130° C.

20. Composition according to claim 1, wherein said at least one structuring polymer has a weight-average molecular mass ranging from 500 to 200,000, preferably ranging from 1,000 to 100,000, more preferably ranging from 2,000 to 30,000.

21. Composition according to claim 1, wherein said composition has a hardness ranging from 30 to 300 gf.

22. Composition according to claim 1, wherein said composition has a hardness ranging 30 to 200 gf.

23. Composition according to claim 1, wherein said at least one liquid fatty phase of the composition comprises at least one oil chosen from at least one polar oil and at least one apolar oil having an affinity with the at least one structuring polymer.

24. Composition according to claim 23, wherein said at least one polar oil is chosen from:
hydrocarbon-based plant oils with a high content of triglycerides comprising fatty acid esters of glycerol in which the fatty acids comprise chains having from 4 to 24 carbon atoms, said chains optionally being chosen from linear and branched, and saturated and unsaturated chains;
synthetic oils or esters of formula R₅COOR₆ in which R₅ is chosen from linear and branched fatty acid residues comprising from 1 to 40 carbon atoms and R₆ is chosen from hydrocarbon-based chain containing form 1 to 40 carbon atoms, with the proviso that R₅+R₆≧10;
synthetic ethers containing from 10 to 40 carbon atoms;
C₈ to C₂₆ fatty alcohols; and
C₈ to C₂₆ fatty acids.

25. Composition according to claim 23, wherein said at least one apolar oil is chosen from:
silicone oils chosen from volatile and non-volatile, linear and cyclic polydimethylsiloxanes that are liquid at room temperature;
phenylsilicones, in particular phenyltrimethicone; and
hydrocarbons chosen from linear and branched, volatile and non-volatile hydrocarbons of synthetic and mineral origin.

26. Composition according to claim 1, wherein said at least one liquid fatty phase is present in an amount ranging from 1% to 99% by weight relative to the total weight of the composition.

27. Composition according to claim 1, wherein said at least one liquid fatty phase is present in an amount ranging from 10% to 80% by weight relative to the total weight of the composition.

28. Composition according to claim 1, wherein said at least one liquid fatty phase comprises at least one volatile solvent chosen from hydrocarbon-based solvents and silicone solvents.

29. Composition according to claim 1, wherein said at least one silicone gum is poly(dimethylsiloxane).

30. Composition according to claim 1, furtgher comprising at least one short chain ester chosen from R₅COOR₆ in which R₅ is chosen from linear and branched fatty acid residues containing from 1 to 18 carbon atoms and R₆ is chosen from, for example, a hydrocarbon-based chain containing from 1 to 18 carbon atoms.

31. Composition according to claim 30, wherein said at least one short chain ester is chosen from isodecyl neopentanoate, ethylhexyl ethylhexanoate, diisopropyl adipate, dicapryl ester, isononyl isononanoate, and their mixtures.

31. Composition according to claim 1, further comprising at least one crystalline silicone compound.

32. Composition according to claim 31, wherein said at least one crystalline silicone compound is present in an amount ranging from 0.05% to 35% by weight relative to the total weight of the composition.

33. Composition according to claim 1, further comprising at least one additional additive chosen from antioxidants, essential oils, preserving agents, fragrances, fillers, waxes, neutralizing agents, gums, liposoluble polymers that are dispersible in the medium, cosmetic and dermatological active agents, dispersants, and an aqueous phase containing water that is optionally thickened or gelled with an aqueous-phase thickener or gelling agent and optionally water-miscible compounds.

34. Composition according to claim 1 further comprising at least one coloring agent.

35. Composition according to claim 1, wherein said at least one coloring agent is chosen from lipophilic dyes, hydrophilic dyes, pigments and nacres.

36. Composition according to claim 35, wherein said at least one coloring agent is present in a proportion of from 0.01% to 50%.

37. Composition according to claim 1, wherein said composition is a solid.

38. Composition according claim 1, wherein said composition is a solid chosen from molded and poured sticks.

39. Composition according to claim 1, wherein said composition is in the form of a rigid gel.

40. Composition according claim 1, wherein said composition further comprises at least one wax.

41. Composition according to claim 41, wherein said at least one wax is chosen from beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters and glycerides that are solid at 40° C.

42. Composition according to claim 1, wherein said composition is in the form of an anhydrous stick.

43. A make-up or care or treatment composition for the skin, the lips, or keratinous fibers containing at least one liquid fatty phase comprising (i) at least one oil structured with at least one structuring polymer as described in claim 1, and (ii) at least one silicone gum, said oil having an affinity with said structuring polymer and/or with the silicone gum, and wherein the liquid fatty phase, the polymer and the silicone gum form a physiologically acceptable medium.

44. A make-up or care or treatment composition for the skin, the lips, or keratinous fibers comprising at least one liquid fatty phase comprising (i) at least one oil structured with at least one structuring polymer as described in claim 1, (ii) at least one silicone gum, and at least one coloring agent, said oil having an affinity with said structuring polymer and/or with the silicone gum, and wherein the liquid fatty phase, the polymer and the silicone gum form a physiologically acceptable medium.

45. A mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises at least one liquid fatty phase comprising (i) at least one oil structured with at least one structuring polymer as described in claim 1, and (ii) at least one silicone gum, said oil having an affinity with said structuring polymer and/or with the silicone gum, and wherein the liquid fatty phase, the polymer and the silicone gum form a physiologically acceptable medium.

46. A lipstick composition comprising an anhydrous composition containing at least one liquid fatty phase comprising (i) at least one oil structured with at least one structuring polymer as described in claim 1, and (ii) at least one silicone gum, said oil having an affinity with said structuring polymer and/or with the silicone gum, and wherein the liquid fatty phase, the polymer and the silicone gum form a physiologically acceptable medium.

47. A method for care, make-up or treatment of keratin materials comprising applying to the keratin materials an anhydrous composition containing at least one liquid fatty phase comprising (i) at I and (ii) at least one silicone gum, said oil having an affinity with said structuring polymer and/or with the silicone gum, and wherein the liquid fatty phase, the polymer and the silicone gum form a physiologically acceptable medium.

48. A method for care, make-up or treatment of keratinous fibers, lips, or skin comprising applying to the keratinous fibers, lips, or skin a composition comprising at least one liquid fatty phase comprising (i) at least one oil structured with at least one structuring polymer described in claim 1, and (ii) at least one silicone gum, said oil having an affinity with said structuring polymer and/or with the silicone gum, and wherein the liquid fatty phase, the polymer and the silicone gum forming a physiologically acceptable medium.

49. A method for providing an anhydrous composition having at least one property chosen from non-exudation, gloss, and comfortable deposit on keratin materials chosen from lips, skin, and keratinous fibers, comprising including in the composition at least one liquid fatty phase comprising (i) at least one oil structured with at least one structuring polymer as described in claim 1, and (ii) at least one silicone gum, said oil having an affinity with said structuring polymer and/or with the silicone gum, and wherein the liquid fatty phase, the polymer and the silicone gum forming a physiologically acceptable medium.

50. A method of making up or caring for skin, lips or keratinous fibers comprising applying to the skin, lips, or keratinous fibers a structured composition containing at least one liquid fatty phase comprising (i) at least one oil structured with at least one structuring polymer as described in claim 1, and (ii) at least one silicone gum, said oil having an affinity with said structuring polymer and/or with the silicone gum, and wherein the liquid fatty phase, the polymer and the silicone gum forming a physiologically acceptable medium.
